(19) United States
(12) Patent Application Publication
(10) Pub. No.: US 2005/0113584 A1
(43) Pub. Date: May 26, 2005

(54) Methods for the preparation of rhodamine

(75) Inventors: Robert D. Damoiseaux, Escondido, CA (US); Jennifer L. Harris, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, HM LX Hamilton (BM)

(21) Appl. No.: 10/891,826

(22) Filed: Jul. 14, 2004

Related U.S. Application Data
(60) Provisional application No. 60/487,331, filed on Jul. 14, 2003.

(51) Int. Cl. C07D 311/88

(52) U.S. Cl. 549/227

(57) ABSTRACT

Method for preparing rhodamine on a solid support and, in particular, methods for the economical preparation of rhodamine NHS ester are disclosed.

### CROSS-REFERENCES TO RELATED APPLICATIONS

This application claims benefit of U.S. Provisional Application No. 60/487,331, filed Jul. 14, 2003, which application is incorporated herein by reference for all purposes.

### BACKGROUND OF THE INVENTION

The attachment of the rhodamine NHS ester to a solid support and use of the rhodamine free amines as attachment points for peptides is especially attractive in peptide chemistry and in screening assays for protease activity. Such a structure can be used as a vehicle for the preparation and facile identification of libraries of peptides. Accordingly, the generation of libraries in a combinatorial split-mix fashion would be economically feasible with a solid support modified with rhodamine. However, the high cost of commercial Rhodamine NHS ester (compound 5) provides a significant barrier to the application of this compound to solid supports for the generation of libraries.

Surprisingly, the present invention provides a new procedure for the preparation of the rhodamine NHS ester and, in turn, the preparation of a solid support comprising the rhodamine. Such rhodamine-modified solid supports would be particularly advantageous in, for example, protease drug screening systems.

### SUMMARY OF THE INVENTION

In one aspect, the present invention provides a method for preparing a rhodamine compound of Formula III having the following structure: 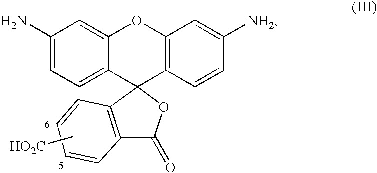
the method comprising: contacting a compound of Formula I having the following structure: 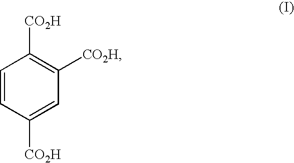
with a condensing agent and at least two equivalents of a compound of Formula II having the following structure: 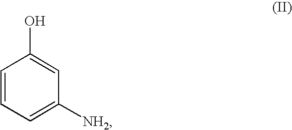
to afford the rhodamine compound of Formula III.

In a second aspect, the present invention provides a method for preparing a rhodamine compound of Formula IV having the following structure: 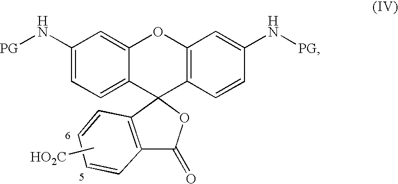
wherein PG is a protecting group; the method comprising: contacting a compound of Formula III having the following structure: 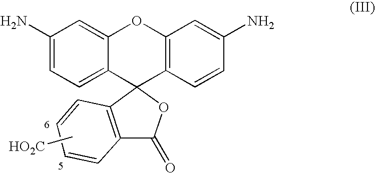
with a protecting group precursor to afford the rhodamine compound of Formula IV.

In a third aspect, the present invention provides a method for preparing a rhodamine modified solid support structure of Formula VI: 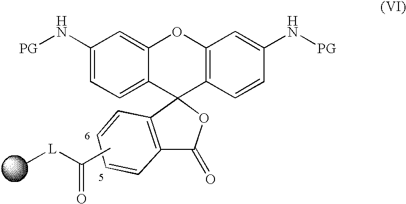
wherein: PG is a protecting group; L is a linker; and 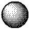
is a solid support structure; the method comprising: a) contacting a compound of Formula III having the following structure: 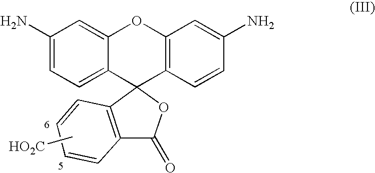
with a protecting group precursor to afford a compound of Formula IV having the following structure: 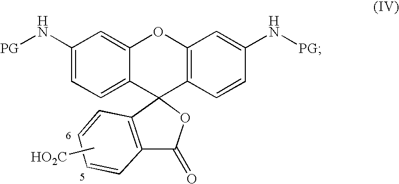
b) contacting the compound of Formula IV with N-hydroxysuccinimide and a first coupling agent to afford a compound of Formula V: 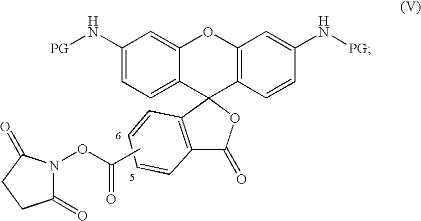
c) contacting the compound of Formula V with a solid support to afford the rhodamine modified solid support structure of Formula VI.

In a fourth aspect, the present invention provides a method for preparing a compound of Formula VII having the following structure: 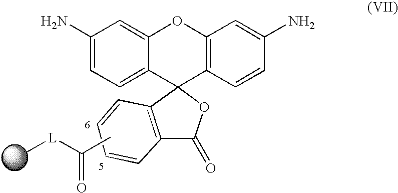
the method comprising contacting a compound of Formula V having the following structure: 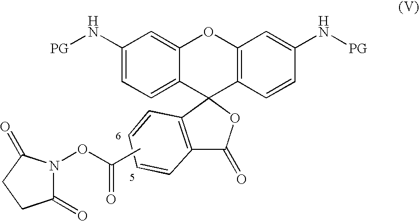
wherein: PG is a protecting group; with a solid support to afford a rhodamine modified solid support of Formula VI having the following structure: 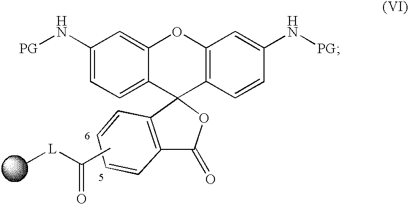
deprotecting the rhodamine modified solid support of Formula VI to afford the compound of Formula VII.

In a fifth aspect, the present invention provides a method for preparing a compound of Formula VIII having the following structure: 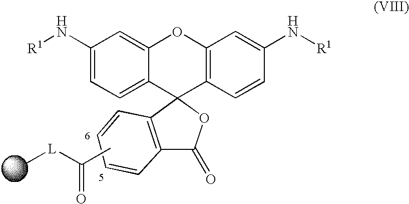
wherein: each R¹ is a member selected from the group consisting of an amino acid, a polypeptide or protein sequence, a nucleotide sequence, a lipid, a carbohydrate and a small organic molecule; L is a linker; and 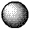
is a solid support;
the method comprising: deprotecting a compound of Formula VI having the following structure: 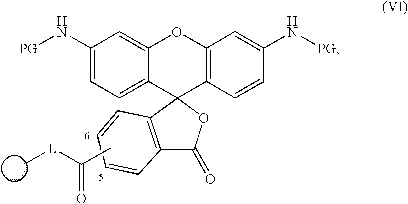
   wherein: PG is a protecting group; to afford a compound of Formula VII having the following structure: 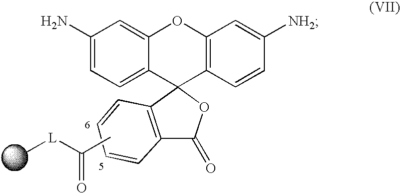
   and
   contacting the compound of Formula VII with a R¹ precursor in the presence of a second base and a second coupling agent to afford the compound of Formula VIII.

In a sixth aspect, the present invention provides a rhodamine compound of Formula III having the following structure:

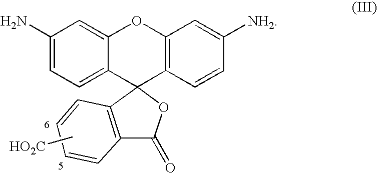

In a seventh aspect, the present invention provides a rhodamine compound of Formula IV having the following structure: 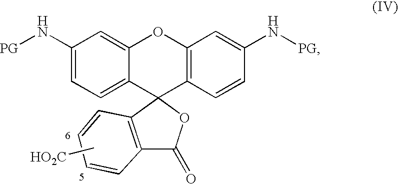
wherein: PG is a protecting group.

In an eighth aspect, the present invention provides a rhodamine modified solid support structure of Formula VI having the following structure: 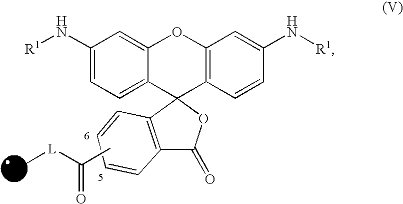
wherein: R¹ is a member selected from the group consisting of a protecting group, an amino acid, a polypeptide or protein sequence, a nucleotide sequence, a lipid, a carbohydrate and a small organic molecule; L is a linker; and 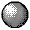
is a solid support.

Other features, objects and advantages of the invention and its preferred embodiments will become apparent from the detained description, examples, claims and figures that follow.

### DETAILED DESCRIPTION OF THE INVENTION AND PREFERRED EMBODIMENTS

I. Definitions

As used herein, the term “amino acid” refers to both natural, non-natural and synthetic amino acids. The natural amino acids used in the present invention are referred to herein by their common single letter abbreviations.

As used herein, the term “condensing agent” refers to a chemical agent that facilitates the reaction of at least two separate chemical species, producing water in the process. Suitable condensing agents include acids, such as sulfuric acid.

As used herein, the term “contacting” refers to the process of bringing into contact at least two distinct species such that they can react. In one embodiment, contacting an amine and an ester under appropriate conditions known to one of skill in the art would result in the formation of an amide.

As used herein, the term “coupling agent” refers to a chemical agent that facilitates the reaction of at least two separate chemical species. Suitable coupling agents include HATU, HOBt, carbodiimides such as EDC, DIC and 3-(3′-dimethylaminopropyl)carbodiimide hydrochloride, among others. One of skill in the art will appreciate that other coupling agents are suitable in the present invention.

As used herein, the term “dehydrating agent” refers to an organic or inorganic compound or substance that removes water, or hydrogen and oxygen in a ratio so as to form water, from a chemical compound, reaction mixture, or solution. Suitable dehydrating agents include, for example, ZnCl₂ smelter. Other dehydrating agents suitable in the present invention will be apparent to one of skill in the art.

As used herein, the term “deprotecting” refers to the process of removing a protecting group to reveal the sensitive or reactive functional group. Suitable methods of deprotecting the protecting groups of the present invention can be found in “Protective Groups in Organic Chemistry,” 3^{rd} ed., T. W. Greene and P. G. M. Wuts, John Wiley & Sons, New York, N.Y., 1999.

As used herein, the term “linker” refers to a chemical moiety that links the rhodamine to the solid support. The linkers of the present invention are optionally cleavable. Linkers of the present invention, include, for example, a Rink amide linker. One of skill in the art will appreciate that other linkers are useful in the present invention.

As used herein, the term “peptide” refers to a compound made up of a single unbranched chain of amino acid residues linked by peptide bonds. The number of amino acid residues in such compounds varies widely. Peptides referred to herein preferably have from 2 to 70 amino acid residues. More preferably, peptides referred to herein have from 2 to 50 amino acid residues.

As used herein, the term “protein” refers to a complex of two or more peptides which can be linked by bonds other than peptide bonds, for example, such peptides making up the protein can be linked by disulfide bonds. Proteins referred to herein usually have from a few tens of amino acid residues, e.g., 20, to up to a few hundred amino acid residues, e.g., 200, or more.

As used herein, the term “protecting group” refers to a chemical moiety that protects a sensitive functional group to a reaction elsewhere in the molecule. Following the reaction, the protecting group is removed to reveal the sensitive functional group. Useful protecting groups are described in Geiger and Konig, 1981, “The Peptides” (Gross and Meinhofer, eds.) pp. 3-101, Academic Press: New York). A very useful combination involves base- and acid-cleavable protecting groups. Many protecting groups useful in the present invention can be found in “Protective Groups in Organic Chemistry,” 3^{rd} ed., T. W. Greene and P. G. M. Wuts, John Wiley & Sons, New York, N.Y., 1999. Other protecting groups useful in the present invention are known to one of skill in the art.

As used herein, the term “protecting group precursor” refers to the chemical agent that provides the suitable protecting group upon reaction with the appropriate sensitive functional group.

As used herein, the term “purifying” refers to the process of removing any side products and undesirable chemical compounds from the desired product. Suitable methods of purifying the compounds of the present invention include chromatography, such as column chromatography and liquid chromatography, and extraction. One of skill in the art will appreciate that further methods of purifying the compounds of the present invention are suitable.

As used herein, the term “small organic molecule” refers to an organic molecules with a molecular weight of less than about 750.

As used herein, the term “solvent system” refers to a mixture of one or more solvents that can further comprise additional reagents.

II. General

The rhodamine compounds of the present invention, such as Rhodamine NHS esters, are fluorescent dyes that are usable in many different applications, such as in the labelling of molecules including, but not limited to, oligonucleotides and proteins. Such fluorescent dyes exhibit a red shifted fluorescence/absorption spectrum which leads to a reduced background and has the advantage of being very photostable, showing nearly no photobleaching. Furthermore, such fluorescent properties are largely independent of the pH. Therefore, the rhodamine compounds of the present invention are superior to most available dyes. Moreover, such rhodamine compounds are very versatile since they can be used with an argon ion laser as a source for excitation which, in turn, allows for the use of, e.g., Affymetrix gene chip scanners.

The use of the rhodamine compounds of the present invention, such as Rhodamine NHS esters, as scaffolds on solid supports allows for the generation of many different compounds in a short period of time. A particular application would be their use in the generation and use of combinatorial split-mix compound libraries. This is very important since the intrinsic properties of the rhodamine scaffolds having small molecules/peptides attached to the amino moieties allow for the use of these conjugates in enzymatic assays, e.g., monitoring proteolytic activities (Leytus et al., Biochem J., 209:299-307 (1983); Hug et al., Biochemistry, 38:13906-11 (1999). Therefore, the rhodamine compounds of the present invention have a significant impact on drug screening of enzymes, especially proteases, making the drug screening process so much easier. The residues attached to the rhodamine scaffold can vary significantly and, in preferred embodiments, include an amino acid, a polypeptide sequence, a nucleotide sequence, a lipid, a carbohydrate and a small organic molecule. In preferred embodiments, the residues attached to the rhodamine scaffold are polypeptide sequences, and the methods of the present invention allow for the possibility of performing solid support peptide synthesis on the rhodamine scaffold.

III. Preferred Embodiments

A. Methods

In one aspect, the present invention provides a method for preparing a rhodamine compound of Formula III having the following structure: 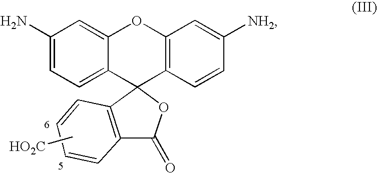
the method comprising: contacting a compound of Formula I having the following structure: 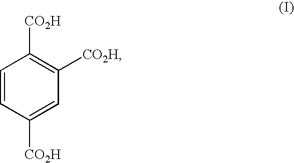
with a condensing agent and at least two equivalents of a compound of Formula II having the following structure: 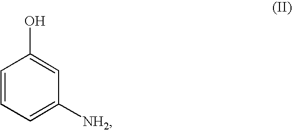
to afford the rhodamine compound of Formula III. In a preferred aspect, the present invention provides a method for preparing a rhodamine compound of Formula III, wherein the condensing agent is an acid. In a more preferred aspect, the present invention provides a method for preparing a rhodamine compound of Formula III, wherein the acid is H₂SO₄. In another preferred aspect, the present invention provides a method for preparing a rhodamine compound of Formula III, wherein the acid is substituted with a dehydrating agent. In a more preferred aspect, the present invention provides a method for preparing a rhodamine compound of Formula III, wherein the dehydrating agent is ZnCl₂ smelter.

In a preferred aspect, the present invention provides a method for preparing a rhodamine compound of Formula III, wherein the contacting is carried out at a temperature of about 160° C. to about 200° C. In a more preferred aspect, the present invention provides a method for preparing a rhodamine compound of Formula III, wherein the contacting is carried out at a temperature of about 180° C. to about 190° C.

In another preferred aspect, the present invention provides a method for preparing a rhodamine compound of Formula III, further comprising purifying the compound of Formula III. In a more preferred aspect, the present invention provides a method for preparing a rhodamine compound of Formula III, wherein purifying the compound of Formula III is carried out using chromatography. Preferred chromatographic methods include liquid chromatography, high-pressure liquid chromatography, reverse-phase chromatography, and column chromatography, for example. One of skill in the art will appreciate that further types of chromatography are useful in the present invention.

In yet another preferred aspect, the present invention provides a method for preparing a rhodamine compound of Formula III, wherein the compound of Formula I is contacted with at least three equivalents of the compound of Formula II.

In another aspect, the present invention provides a method for preparing a rhodamine compound of Formula IV having the following structure: 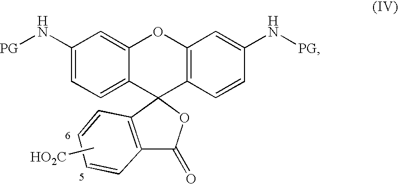
wherein PG is a protecting group; the method comprising: contacting a compound of Formula III having the following structure: 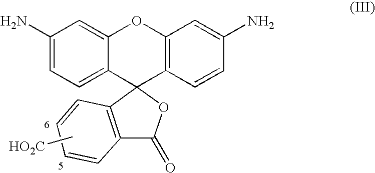
with a protecting group precursor to afford the rhodamine compound of Formula IV. In a preferred aspect, the present invention provides a method for preparing a rhodamine compound of Formula IV, wherein the protecting group precursors are those suitable for protecting amines. Suitable protecting groups and protecting group precursors can be found in “Protective Groups in Organic Synthesis,” T. W. Greene and P. G. M. Wuts, 3^{rd} ed., 1999. One of skill in the art will appreciate that other protecting groups are also suitable for use in the present invention.

In a preferred aspect, the present invention provides a method for preparing a rhodamine compound of Formula IV, wherein the protecting group precursor is a member selected from the group consisting of trifluoroacetic acid and 4,4′-dimethoxytrityl. In a more preferred aspect, the present invention provides a method for preparing a rhodamine compound of Formula IV, wherein the protecting group precursor is trifluoroacetic acid.

In another preferred aspect, the present invention provides a method for preparing a rhodamine compound of Formula IV, wherein the contacting is carried out in a solvent system comprising a first base. Bases that are useful in the present invention include pyridine, triethylamine, dimethylformamide, and N-methylpyrrolidinone, for example. In a more preferred aspect, the present invention provides a method for preparing a rhodamine compound of Formula IV, wherein the solvent system comprises pyridine.

In a further aspect, the present invention provides a method for preparing a rhodamine modified solid support structure of Formula VI: 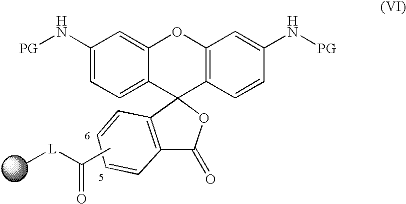
wherein PG is a protecting group; L is a linker; and 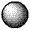
is a solid support structure; the method comprising:
a) contacting a compound of Formula III having the following structure: 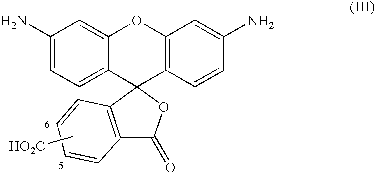
   with a protecting group precursor to afford a compound of Formula IV having the following structure: 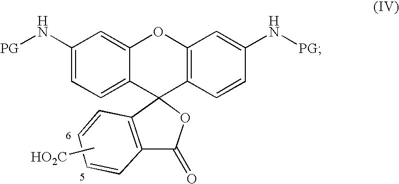
b) contacting the compound of Formula IV with N-hydroxysuccinimide and a first coupling agent to afford a compound of Formula V: 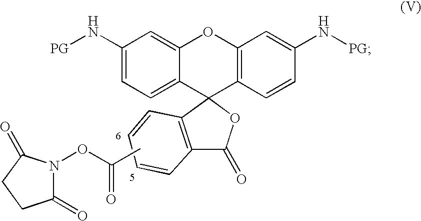
   and
c) contacting the compound of Formula V with a solid support to afford the rhodamine modified solid support structure of Formula VI.

In a preferred aspect, solid supports include silica gels, resins, derivatized plastic films, glass beads, cotton, plastic beads, alumina gels, polysaccharides such as Sepharose and the like, etc. A suitable solid support can be selected on the basis of desired end use and suitability for various synthetic protocols. For example, in polyamide synthesis, useful solid phase support can be resins such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), Rink amide resin, polydimethyl-acrylamide resin (available from Milligen/Biosearch, California), or PEGA beads (obtained from Polymer Laboratories). A preferred solid support also has reactive functional groups, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching a linker which contains one or more reactive groups for the attachment of the rhodamine unit.

In another preferred aspect, a linker is any molecule containing a chain of atoms, e.g., carbon, nitrogen, oxygen, sulfur, etc., that serves to link the molecules to be synthesized on the solid support, with the solid support. The linker is usually attached to the support via a covalent bond, before synthesis on the support starts, and provides one or more sites for attachment of precursors of the molecules to be synthesized on the solid support.

In a further preferred aspect, the present invention provides a method for preparing a rhodamine compound of Formula VI, further comprising deprotecting the compound of Formula VI to afford a compound of Formula VII having the following formula:

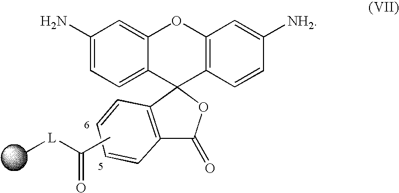

In a more preferred aspect, the present invention provides a method for deprotecting the compound of Formula VI to afford a compound of Formula VII, wherein the deprotecting is carried out using a member selected from the group consisting of ammonia and trichloroacetic acid. One of skill in the art will appreciate that the deprotecting can be carried out via other means (see, “Protective Groups in Organic Chemistry,” 3^{rd} ed., T. W. Greene and P. G. M. Wuts, John Wiley & Sons, New York, N.Y., 1999).

In yet another preferred aspect, the present invention provides a method for preparing a rhodamine compound of Formula VI, wherein the first coupling agent is a member selected from the group consisting of a carbodiimide, 3-(3′-dimethylaminopropyl)carbodiimide hydrochloride and O-(7-azabenzotriazol-1-yl)-N,N,N′,N′-tetramethyluronium hexafluorophosphate. Additional carbodiimides suitable in the present invention include EDC and DIC, for example. One of skill in the art will appreciate that other coupling agents are useful in the present invention.

In still yet another preferred aspect, the present invention provides a method for preparing a rhodamine compound of Formula VI, wherein the solid support is a Rink amide resin.

In yet another aspect, the present invention provides a method for preparing a compound of Formula VII having the following structure: 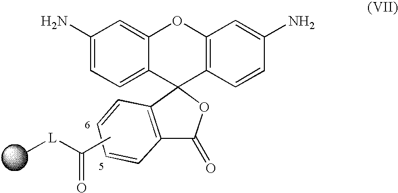
the method comprising contacting a compound of Formula V having the following structure: 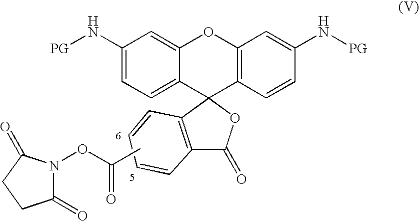
   wherein PG is a protecting group; with a solid support to afford a rhodamine modified solid support of Formula VI having the following structure: 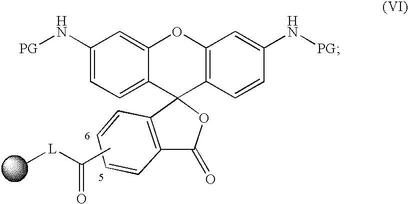
   and
   deprotecting the rhodamine modified solid support of Formula VI to afford the compound of Formula VII. In a preferred aspect, the present invention provides a method for preparing a compound of Formula VII, wherein the deprotecting is carried out using a member selected from the group consisting of ammonia and trichloroacetic acid. One of skill in the art will appreciate that the deprotecting can be carried out via other means (see, “Protective Groups in Organic Synthesis,” T. W. Greene and P. G. M. Wuts, 3^{rd} ed., 1999).

In another preferred aspect, the present invention provides a method for preparing a compound of Formula VII, wherein the solid support is a Rink amide resin.

In still another aspect, the present invention provides a method for preparing a compound of Formula VIII having the following structure: 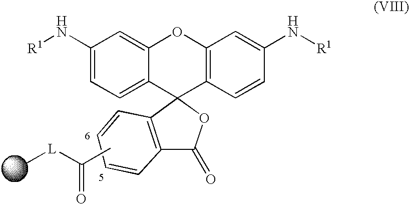
wherein: each R¹ is a member selected from the group consisting of an amino acid, a polypeptide sequence, a nucleotide sequence, a lipid, a carbohydrate and a small organic molecule; L is a linker; and 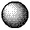
is a solid support;
the method comprising: deprotecting a compound of Formula VI having the following structure: 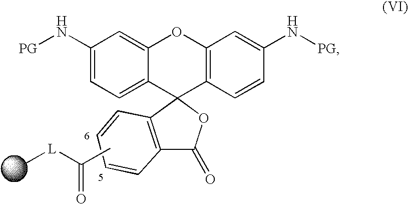
wherein: PG is a protecting group; to afford a compound of Formula VII having the following structure: 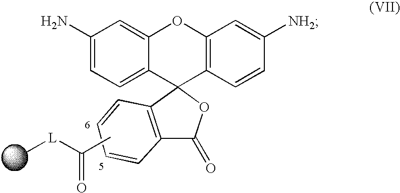
and
contacting the compound of Formula VII with a R¹ precursor in the presence of a second base and a second coupling agent to afford the compound of Formula VIII. In a preferred aspect, the present invention provides a method for preparing a compound of Formula VIII, wherein the R¹ precursor is a member selected from the group consisting of an amino acid, a polypeptide sequence, a nucleotide sequence, a lipid, a carbohydrate and a small organic molecule. In a more preferred aspect, the present invention provides a method for preparing a compound of Formula VIII, wherein the R¹ precursor is an amino acid.

In another preferred aspect, the present invention provides a method for preparing a compound of Formula VIII, wherein the solid support is a Rink amide resin.

In a further preferred aspect, the present invention provides a method for preparing a compound of Formula VIII, wherein the deprotecting is carried out using a member selected from the group consisting of ammonia and trichloroacetic acid.

In still another preferred aspect, the present invention provides a method for preparing a compound of Formula VIII, wherein the second coupling agent is a member selected from the group consisting of HATU, a carbodiimide and O-(7-azabenzotriazol-1-yl)-N,N,N′,N′-tetramethyluronium hexafluorophosphate. Additional carbodiimides suitable in the present invention include EDC and DIC, for example.

In yet another preferred aspect, the present invention provides a method for preparing a compound of Formula VIII, wherein the second base is an organic non-nucleophilic base. In a more preferred aspect, the present invention provides a method for preparing a compound of Formula VIII, wherein the second base is a member selected from the group consisting of collidine, lutidine, quinuclidine, diisopropylamine, triethylamine and diisopropylethylamine. One of skill in the art will appreciate that other amines are useful in the present invention.

B. Compounds

In another aspect, the present invention provides a rhodamine compound of Formula III having the following structure:

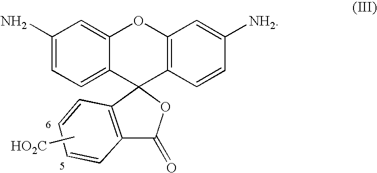

In a further aspect, the present invention provides a rhodamine compound of Formula IV having the following structure: 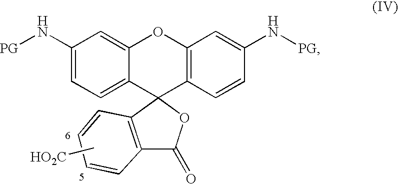
wherein PG is a protecting group. Protecting groups useful in the present invention can be found in “Protective Groups in Organic Synthesis,” T. W. Greene and P. G. M. Wuts, 3^{rd} ed., 1999. One of skill in the art will appreciate that other protecting groups are useful in the present invention.

In yet another aspect, the present invention provides a rhodamine modified solid support structure of Formula VI having the following structure: 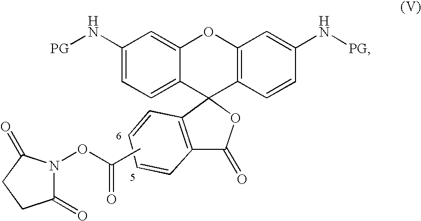
wherein: R¹ is a member selected from the group consisting of a protecting group, an amino acid, a polypeptide sequence, a nucleotide sequence, a lipid, a carbohydrate and a small organic molecule; L is a linker; and 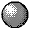
is a solid support. Protecting groups, linkers, and solid supports useful for the rhodamine modified solid support structures of Formula VI are described above.

Once prepared, the rhodamine modified solid supports can be used, for example, in methods for assaying or screening for the presence of enzymatically active enzymes in a sample, such as a biological sample. Suitable assay methods are disclosed in U.S. Provisional Patent Application No. 60/487,464, entitled “FLUOROGENIC ENZYME SUBSTRATES AND USES THEREOF,” filed on Jul. 14, 2003, and bearing Attorney Docket No. 021288-000400, the teachings of which are incorporated herein by reference.

### IV. EXAMPLES

### Example 1

### Preparation of Rhodamine 3

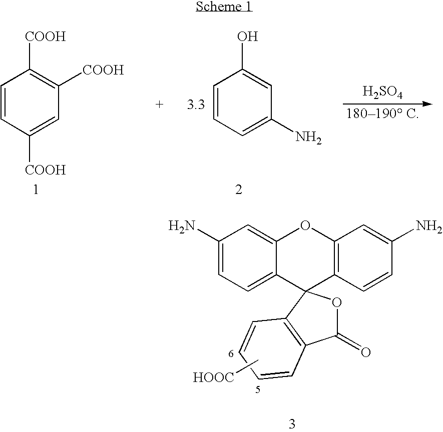

100 g 2 (0.91 mol, 3.3 eq.) were dissolved in 650 mL H₂SO₄ (95-97%) by stirring. 57.6 g 1 (0.27 mol, 1 eq.) were added and dissolved by stirring. The stirred solution was warmed to 180° C. and kept at this temperature for 6 hours. The cool reaction mixture was poured onto 700 g ice and stirred. The sulphuric acid was neutralized with sodium carbonate and 3 L methanol were added to precipitate the inorganic salts. The inorganic salts were removed by filtration. The filter cake was washed with 2 L methanol and the methanolic solutions were unified. After removal of the solvents, the residue was dissolved in methanol and adsorbed onto 300 g silica. Rhodamine 3 was purified by chromatography on 2 kg silica using a step gradient using 35 L of acetonitrile/methanol (7:3) and then 15 L of acetonitrile/methanol/water/triethylamine (20:5:4:1). Removal of the solvents under reduced pressure yielded 65 g 3 (0.17 mol, 63%) as dark red crystalline solid. LC/MS characterization indicated about 90% purity and a 1:1 ratio of the 5/6-isomers of 3.

### Example 2

### Preparation of Rhodamine-NHS Ester

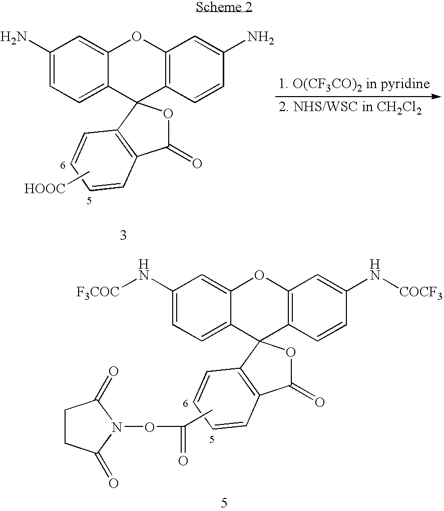

188 mg 3 (0.5 mmol, 1 eq.) were coevaporated three times with 1 mL dry pyridine and suspended in 4 mL dry pyridine. 190 mL trifluoroacetic acid anhydride (1.3 mmol, 2.6 eq) were added dropwise. The reaction mixture was stirred over night and the pyridine was removed under reduced pressure on the next morning. The residue containing the TFA protected compound, 4, was dissolved in 2 mL CH₂Cl₂ and 403 mg N-hydroxysuccinimide (3.5 mmol, 7 eq.) and 477 mg 1-ethyl-3-(3′-dimethylaminopropyl)carbodiimide hydrochloride (2.5 mmol, 5 eq.) were added. The reaction mixture was stirred for 35 min and transferred to a separation funnel with 100 mL CH₂Cl₂ and 100 mL water. The organic phase was dried with sodium sulfate, filtered and the solvent was removed under reduced pressure. Column chromatography on 20 g silica using a step gradient of hexane/ethyl acetate from 75:25 to 45:55 afforded 46 mg 5 (0.07 mmol, 14% over 2 steps).

### Example 3

### Preparation of Rhodamine Rink Resin

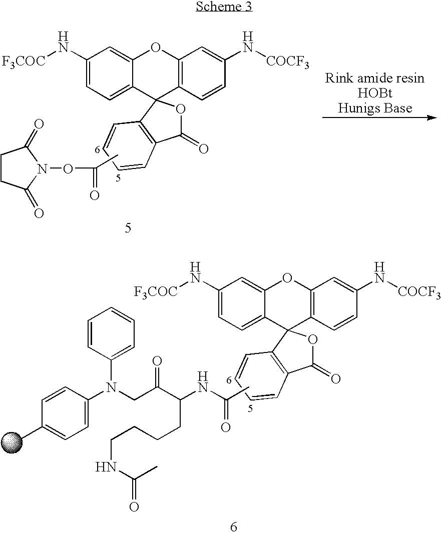

50 mg Rink amide-Lys(Mtt)-Fmoc were washed with DMF (3×, 5 mL, 20 min each) and dichloromethane (3×, 5 mL, 20 min each). The Mtt group was cleaved off (dichloromethane, TFA and TIS at 94:1:5, 2 mL, 2 min reaction time, 4×) and the resin was washed with DMF (3×, 5 mL, 20 min each). The free-amino function was acetylated using acetic acid, HOBt and DICI in DMF (0.3-M each, 3 mL, 1 hour reaction time). The resin was washed with DMF as above and the Fmoc protection group was removed using 20% (v/v) piperidine in DMF (5 mL, 1 hour reaction time). The resin was washed with DMF as above and a solution of 80 mg 5 with 18.4 mg HOBt and 21 μL Hunigs base in DMF (500 ml) was added. After 90 min the resin was washed with DMF as above.

### Example 4

### Deprotection of Rhodamine-Rink Resin and Addition of Amino Acids

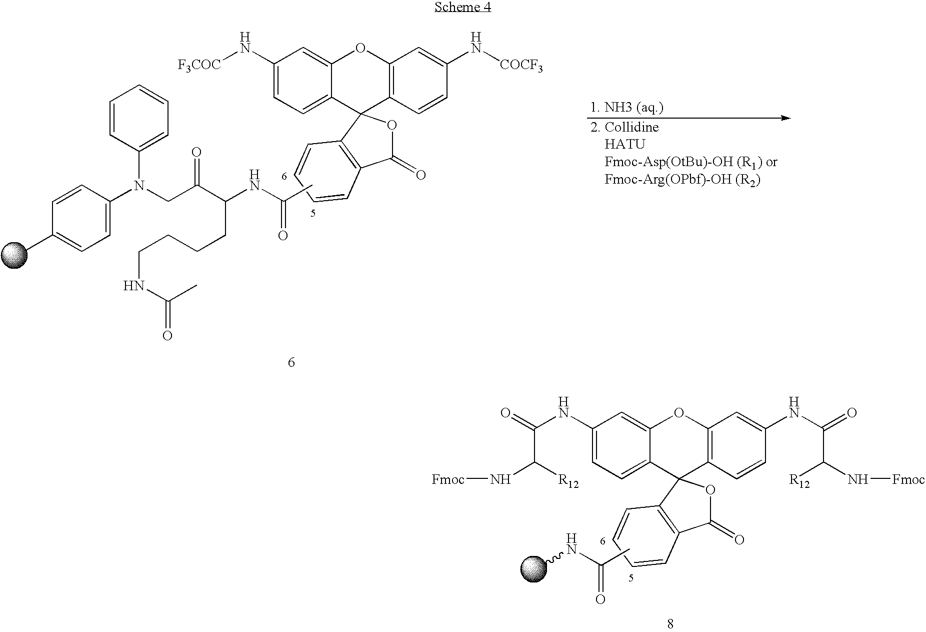

The TFA groups were removed from the rhodamine-rink resin 6 using 5 mL aqueous ammonia. The ammonia was removed after 4 hours and the resin was washed with DMF (15 min, 5 mL, 5×). For the proof of principle, aspartic acid or arginine were coupled for 24 hours to the resin using HATU and collidine (0.5-M amino acid, HATU and collidine). As assayed by LC/MS, the aspartic acid coupled quantitatively, but for the arginine coupling a second and a third subjection were necessary. The resin was washed with DMF (5 mL, 20 min, 3×). After the couplings, possible remaining free rhodamine amino functions were acetylated using acetic acid, DICI and 3-nitrotriazole (1-M each in DMF). The resin was washed as above.

### Example 5

### Further Addition of Amino Acids to Rhodamine-Rink Resin

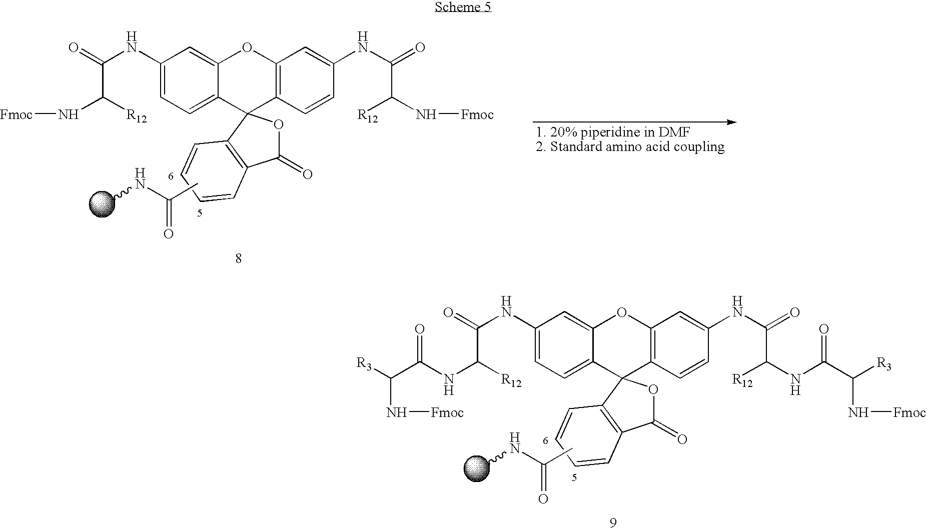

The amino protecting Fmoc group was removed using 20% piperidine in DMF (5 mL, 20 min, 2×). The resin was washed with DMF (5 mL, 15 min, 4×) and the next amino acid was coupled to the resin (amino acid, DICI, HOBT, 0.3-M each, 3 ml). For the following amino acids the same deprotection and coupling conditions were used. On the rhodamine resin modified with arginine, the amino acid sequence nTP (where “n” represents the unnatural amino acid norleucine) was built and on the rhodamine resin modified aspartic acid the amino acid sequence DEV was built, resulting in the sequences (nTPR)₂-rhodamine and (DEVD)₂-rhodamine, respectively. Table I gives an overview of the amino acid quantities coupled to the rhodamine as determined by Fmoc quantification. Finally, the peptides were acetylated using acetic acid, DICI and HOBt (0.3-M each in DMF, 3 mL, 1 hour).

### Example 6

### Cleavage of the Rhodamine-Peptides from the Rhodamine-Rink Resin

After the synthesis, the resin was washed with dichloromethane and dried. The rhodamine-peptides were cleaved of the resin using a cleavage cocktail of TFA, water and TIS (95: 2.5: 2.5) for 1 hour. The solutions were concentrated under reduced pressure to 3 mL and split into three. 1 mL was precipitated in 40 mL diethyl ether, 1 mL was dried directly under reduced pressure and 1 mL was added to 5 mL 20% acetonitrile in water, frozen in liquid nitrogen and lyophilized. The precipitations of the (nTPR)₂-rhodamine and (DEVD)₂-rhodamine yielded 7.5 and 8.5 mg respectively. That corresponds to 4.7 or 5.5 mmoles for the precipitates giving a total of 14.4 and 16.5 mmoles, respectively. These values correspond to the values measured for the Fmoc-piperidine adduct. (Table 1) The identity of the molecules synthesized was confirmed by different means as e.g., mass spectrometry.

TABLE 1
Coupling efficiency for the tetrapeptide synthesis. Fmoc-piperidine adduct
measured after each coupling step in μmoles.

| | DEVD | nTPR |
|---|---|---|
| Coupling No. | (μmoles) | (μmoles) |
| 1 | 37 | 32 |
| 2 | 39 | 34 |
| 3 | 37 | 32 |
| 4 | 37 | 27 |

Although the foregoing invention has been described in some detail by way of illustration and example for purposes of clarity of understanding, it will be obvious that certain changes and modifications may be practiced within the scope of the appended claims. In addition, each reference provided herein is incorporated by reference in its entirety to the same extent as if each reference was individually incorporated by reference.

1. A method for preparing a rhodamine compound of Formula III having the following structure: 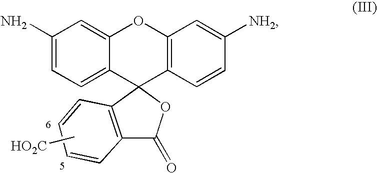
said method comprising:
contacting a compound of Formula I having the following structure: 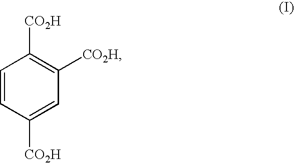
with a condensing agent and at least two equivalents of a compound of Formula II having the following structure: 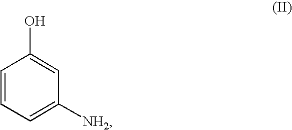
to afford the rhodamine compound of Formula III.

2. The method of claim 1, wherein said condensing agent is an acid.

3. The method of claim 2, wherein said acid is H₂SO₄.

4. The method of claim 3, wherein said acid is substituted with a dehydrating agent.

5. The method of claim 4, wherein said dehydrating agent is ZnCl₂ smelter.

6. The method of claim 1, wherein said contacting is carried out at a temperature of about 160° C. to about 200° C.

7. The method of claim 6, wherein said contacting is carried out at a temperature of about 180° C. to about 190° C.

8. The method of claim 1, further comprising purifying said compound of Formula III.

9. The method of claim 8, wherein purifying said compound of Formula III is carried out using chromatography.

10. The method of claim 1, wherein said compound of Formula I is contacted with at least three equivalents of said compound of Formula II.

11. A method for preparing a rhodamine compound of Formula IV having the following structure: 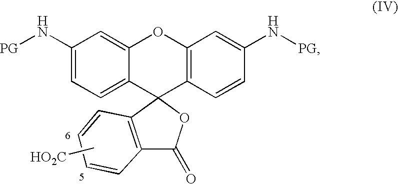
wherein:
PG is a protecting group;
said method comprising:
contacting a compound of Formula III having the following structure: 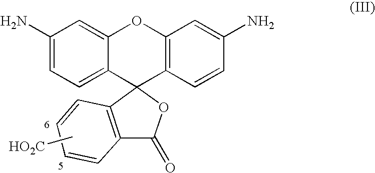
with a protecting group precursor to afford the rhodamine compound of Formula IV.

12. The method of claim 11, wherein said protecting group precursor is a member selected from the group consisting of trifluoroacetic acid and 4,4′-dimethoxytrityl.

13. The method of claim 12, wherein said protecting group precursor is trifluoroacetic acid.

14. The method of claim 11, wherein said contacting is carried out in a solvent system comprising a first base.

15. The method of claim 14, wherein said solvent system comprises pyridine.

16. A method for preparing a rhodamine modified solid support structure of Formula VI: 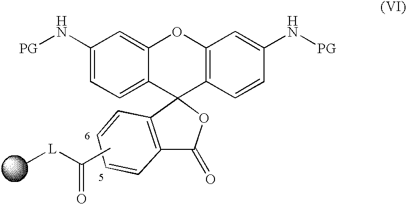
wherein:
PG is a protecting group;
L is a linker; and 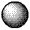
is a solid support structure;
said method comprising:
a) contacting a compound of Formula III having the following structure: 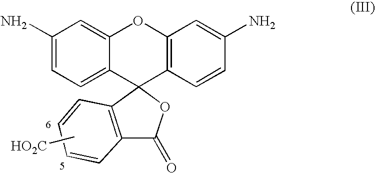
with a protecting group precursor to afford a compound of Formula IV having the following structure: 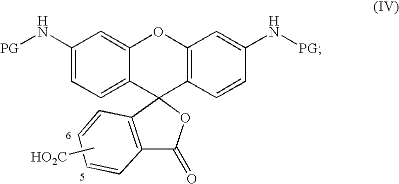
b) contacting said compound of Formula IV with N-hydroxysuccinimide and a first coupling agent to afford a compound of Formula V: 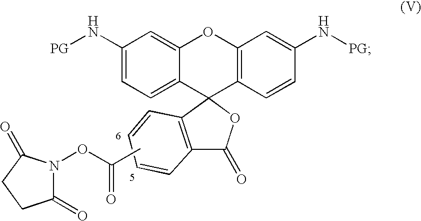
and
c) contacting said compound of Formula V with a solid support to afford said rhodamine modified solid support structure of Formula VI.

17. The method of claim 16, further comprising deprotecting the compound of Formula VI to afford a compound of Formula VII having the following structure:

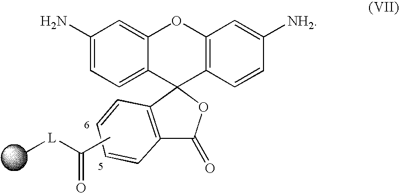

18. The method of claim 17, wherein said deprotecting is carried out using a member selected from the group consisting of ammonia and trichloroacetic acid.

19. The method of claim 16, wherein said first coupling agent is a member selected from the group consisting of a carbodiimide, 3-(3′-dimethylaminopropyl)carbodiimide hydrochloride and O-(7-azabenzotriazol-1-yl)-N,N,N′,N′-tetramethyluronium hexafluorophosphate.

20. The method of claim 16, wherein said solid support is a Rink amide resin.

21. A method for preparing a compound of Formula VII having the following structure: 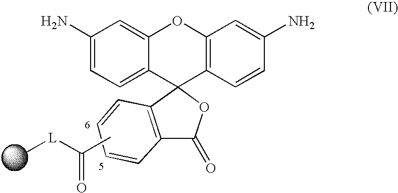
said method comprising:
contacting a compound of Formula V having the following structure: 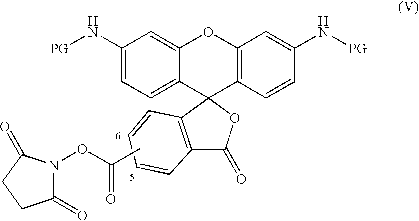
wherein:
PG is a protecting group;
with a solid support to afford a rhodamine modified solid support of Formula VI having the following structure: 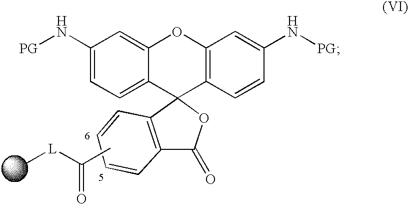
deprotecting said rhodamine modified solid support of Formula VI to afford the compound of Formula VII.

22. The method of claim 21, wherein said deprotecting is carried out using a member selected from the group consisting of ammonia and trichloroacetic acid.

23. The method of claim 21, wherein said solid support is a Rink amide resin.

24. A method for preparing a compound of Formula VIII having the following structure: 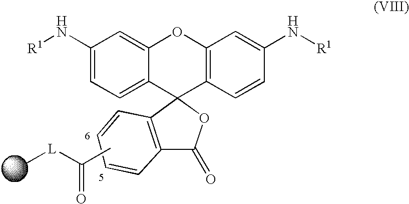
wherein:
each R¹ is a member selected from the group consisting of an amino acid, a peptide, a protein and a small organic molecule;
L is a linker; and 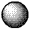
is a solid support;
said method comprising:
deprotecting a compound of Formula VI having the following structure: 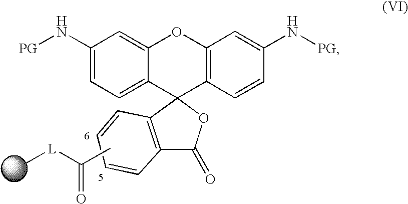
wherein:
PG is a protecting group;
to afford a compound of Formula VII having the following structure: 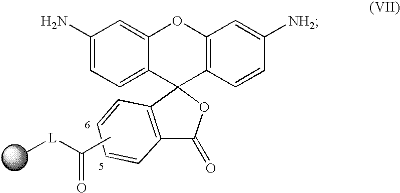
and
contacting said compound of Formula VII with a R¹ precursor in the presence of a second base and a second coupling agent to afford the compound of Formula VIII.

25. The method of claim 24, wherein said R¹ precursor is a member selected from the group consisting of an amino acid, a polypeptide sequence, a nucleotide sequence, a lipid, a carbohydrate and a small organic molecule.

26. The method of claim 25, wherein said R¹ precursor is an amino acid.

27. The method of claim 24, wherein said solid support is a Rink amide resin.

28. The method of claim 24, wherein said deprotecting is carried out using a member selected from the group consisting of ammonia and trichloroacetic acid.

29. The method of claim 24, wherein said second coupling agent is a member selected from the group consisting of HATU, a carbodiimide and O-(7-azabenzotriazol-1-yl)-N,N,N′,N′-tetramethyluronium hexafluorophosphate.

30. The method of claim 24, wherein said second base is a non-nucleophilic base.

31. The method of claim 30, wherein said second base is a member selected from the group consisting of collidine, lutidine, quinuclidine, diisopropylamine, triethylamine and diisopropylethylamine.

32. A rhodamine compound of Formula III having the following structure:

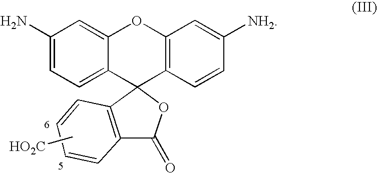

33. A rhodamine compound of Formula IV having the following structure: 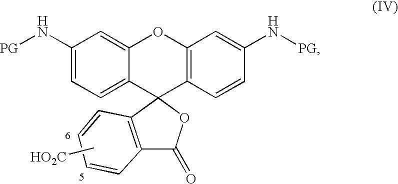
wherein:
PG is a protecting group.

34. A rhodamine modified solid support structure of Formula VI having the following structure: 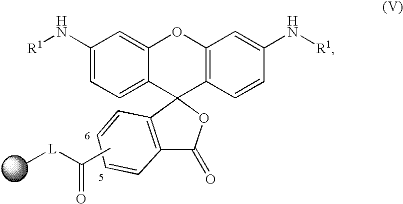
wherein:
R¹ is a member selected from the group consisting of a protecting group, an amino acid, a polypeptide sequence, a nucleotide sequence, a lipid, a carbohydrate and a small organic molecule;
L is a linker; and 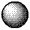
is a solid support.
